SEQUENCE LISTING (5 sequences)

SEQ ID NO: 1 - DNA, 1883 bp, Homo sapiens
  gene (1)..(1883): TGF-beta RII
cccatcaaagaagttatgattcaatccacgaagaccaggagttggcgaaatgaagaaaaaaaggtcagaggaaggaagtcctctctggggaaggctctaagcataaagggcaggaggattacagaggcatatctcgaaatttggagaaggctttcagtaagcaaggagaagccaaatgaaagtttacggagagttggaggcttgaagacaccgttcaaggatctggtttttatcttctctttattctcaagagcttagtgggaagccattaaatgattttaatcaaggaggggttggttataaactagttttgttaattttgaaaaatctgaattcactctcgtttgagaaactgagtgaaagagcccagaacggccgtgctgagggtgactcctgggaagactccttaaccacaagccatggcagtggcatgggctggtggcagaagagggaatagggagaagatttggaactcaatcttcctccattgacaaagtcactccagctttggcaaggcaattaattggtgggaaagaagatgcctagccctcctgatttcactgcactttctgcatcttcaacatgagtactgggaagtggcaaaacaatccagaggcaggcttgggtgctaggtggagcatgagttaaaattccaggatgaagcaaatgaacacttagaatgacaggaaagatttgggagttgggtttgggggagggctatttacctttattccctggagaccctggcacaaaccctgcctctgcaatcttcctctcaggtaaaggaattcattaaatgaattgctagaagatctactgaccagagggctgtacagaatcatatctttgagagtgggaagtaggttgatcacatagtttattatccaatcaggacatatctgaaagagaaagggggttctattaatatttaaactacaaaacatgtacaccaggaatgtcttgggcaaatctggttgccctagcaagaaaggaaatttgaaagtttatgctgttctgctcccatgttaccccgtttgcacatgagagggtaagtattctctttcttcacctgcattaagggaataaaagcacaagcattcaggtgactcccaacccacttttaattttacagtttctgctatactctatacattctgaaaattacatttcccaccactatacttcgtgataggtgatcatttacaattactcactgactcagtcccgggaagaggcggtgcaaaatggacgctctatccaggtgctcattagaaatgcagaatctctgcctgcctcctagacctactgaattagaatctgcatttttaaataagatttccaggtgatcaatatgtacattaaaacttgagaaaaacctctagacttcgacctaaagaaaaacattttacaacttgacagtgtatgcacatacatacatgcatatagacacaactgaagcacaaatttaatgaagtagaatttaccgttactattttatttggaaagaaatgtgctcgcgactcaatagattggagtattcactcctggatctcaacttgcaatttgaaaacgcatctctaaagcacctaggagcaatctgaagaaagctgaggggaggcggcagatgttctgatctactagggaaaacgtggacgttttctgttgttactttgtgaactgtgtgcacttagtcattcttgagtaaatacttggagcgaggaactcctgagtggtgtgggagggcggtgaggggcagctgaaagtcggccaaagctctcggaggggctggtctaggaaacatgattggcagctacgagagagctaggggctggacgtcgaggagagggagaaggctctcgggcggagagaggtcctgcccagct

SEQ ID NO: 2 - DNA, 25 bp, Artificial Sequence
  Primer
ggacatatctgaaagagaaaggggg

SEQ ID NO: 3 - DNA, 22 bp, Artificial Sequence
  Primer
ttgggagtcacctgaatgcttg

SEQ ID NO: 4 - DNA, 15 bp, Artificial Sequence
  Primer_bind
gtnrccnnnrgyaac

SEQ ID NO: 5 - DNA, 18 bp, Artificial Sequence
  Primer_bind
nngttrcynnngynacnn

(19) United States
(12) Patent Application Publication
(10) Pub. No.: US 2005/0009016 A1
(43) Pub. Date: Jan. 13, 2005

(54) Tgfbeta-rII promoter polymorphisms

(76) Inventors: David W. Moskowitz, Ithaca, NY (US); Lee A. Henderson, Ithaca, NY (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/275,098

(22) PCT Filed: May 4, 2001
(86) PCT No.: PCT/US01/14645

Related U.S. Application Data
(60) Provisional application No. 60/201,813, filed on May 4, 2000.

(51) Int. Cl. C12Q 1/68; C07H 21/04; C07K 14/475

(52) U.S. Cl. 435/6; 435/69.4; 435/320.1; 435/325; 530/399; 536/23.5

(57) ABSTRACT

Disclosed are single nucleotide polymorphisms (SNPs) associated with end stage renal disease, breast cancer, lung cancer, and prostate cancer. Also disclosed are methods for using SNPs to determine susceptibility to these diseases; nucleotide sequences containing SNPs; kits for determining the presence of SNPs; and methods of treatment or prophylaxis based on the presence of SNPs.

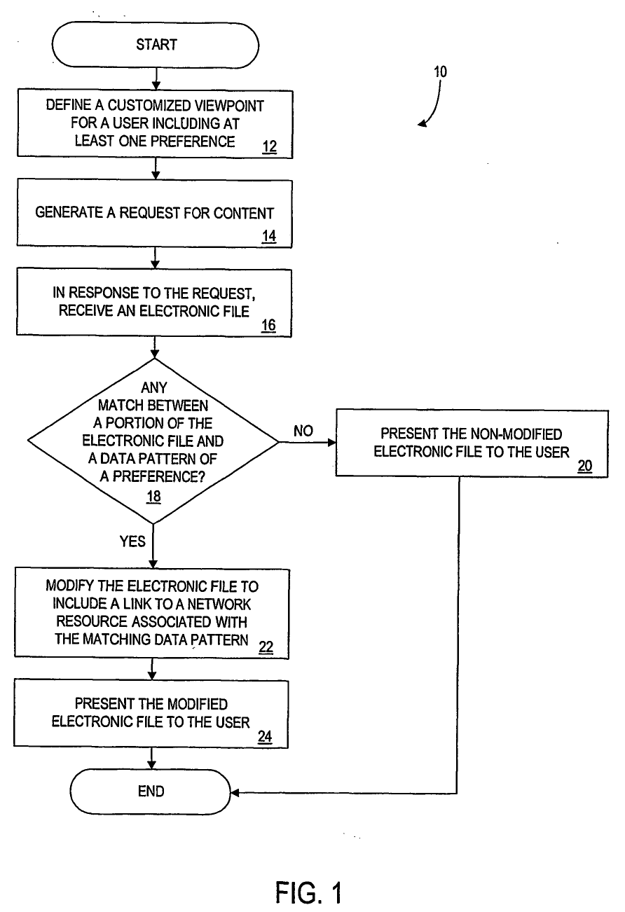

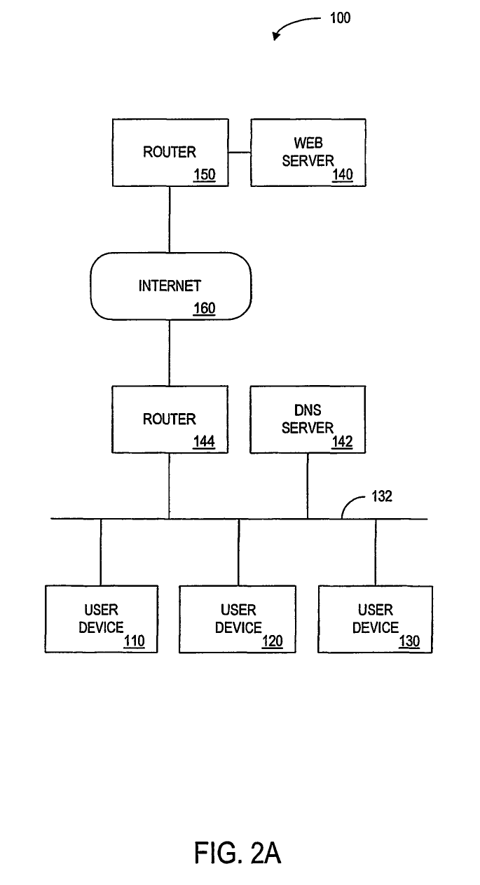

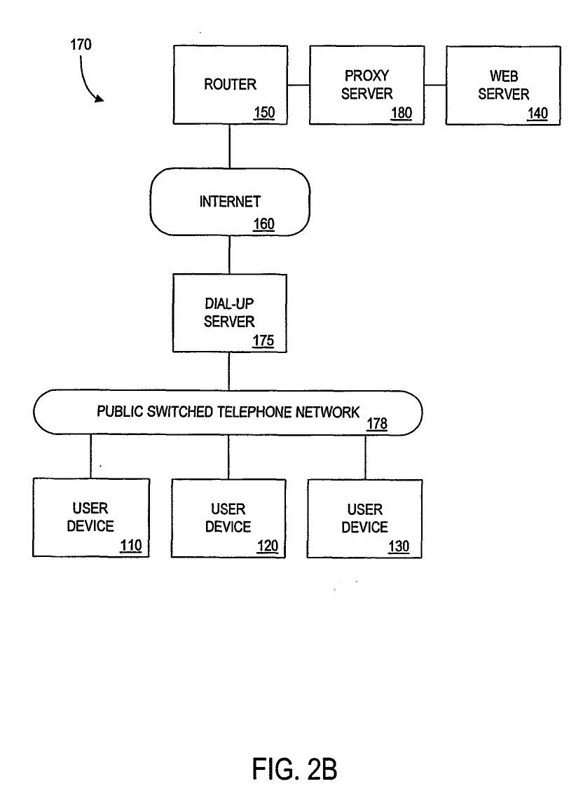

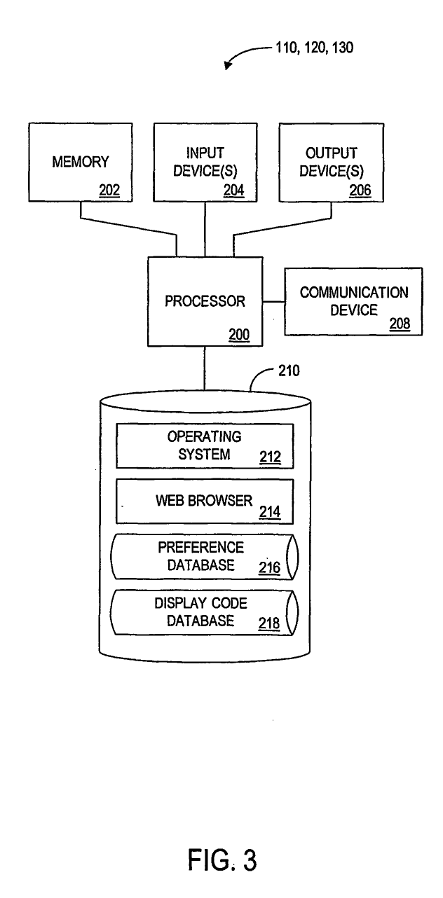

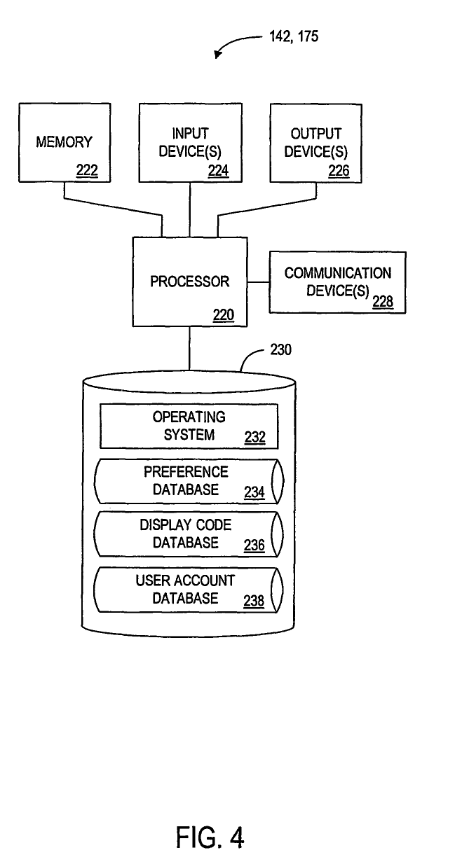

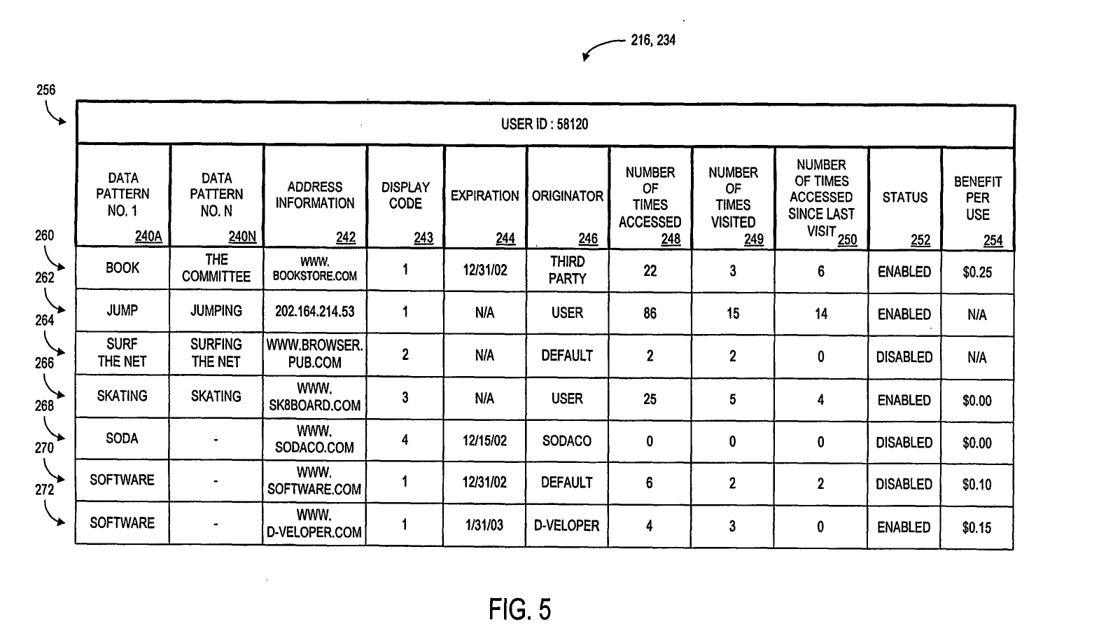

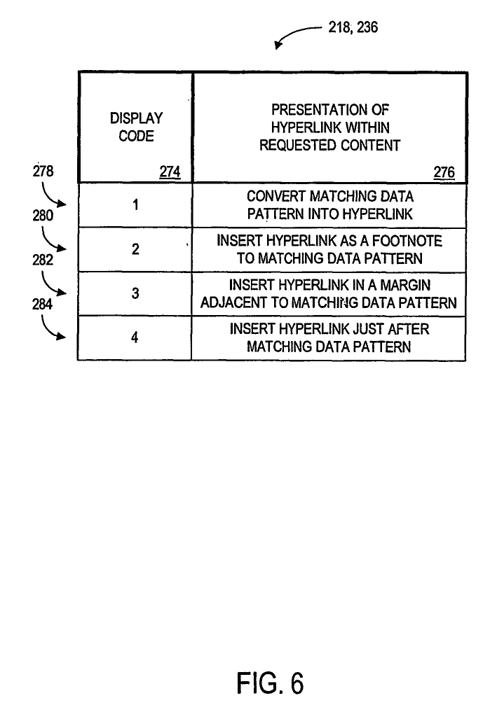

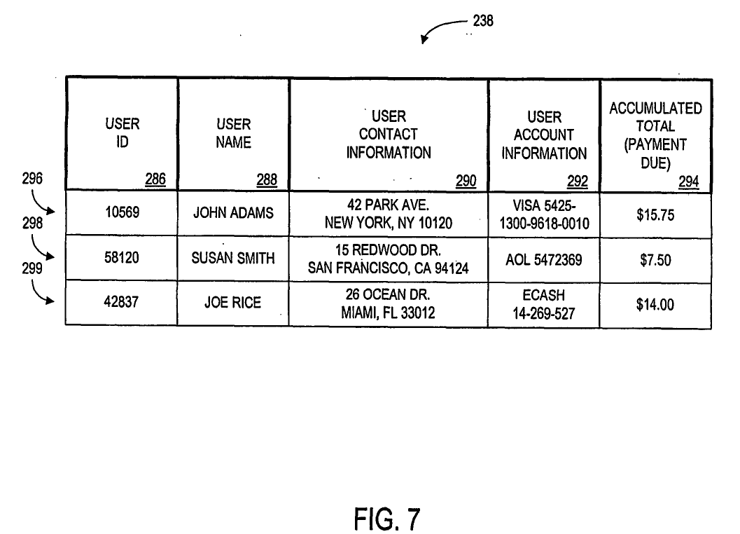

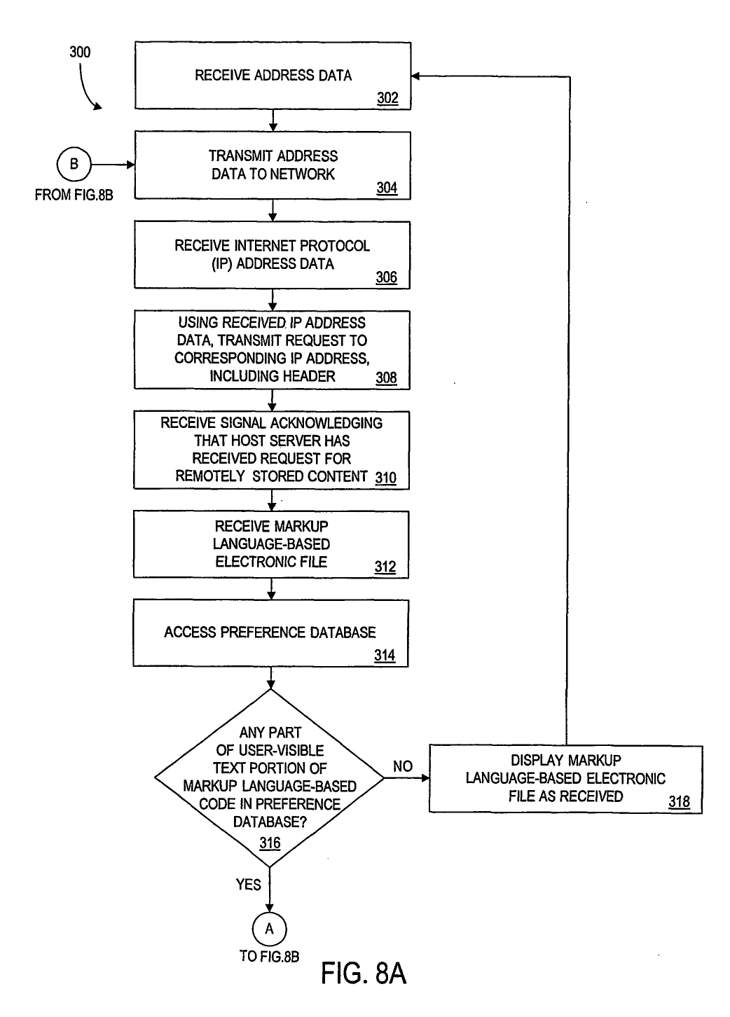

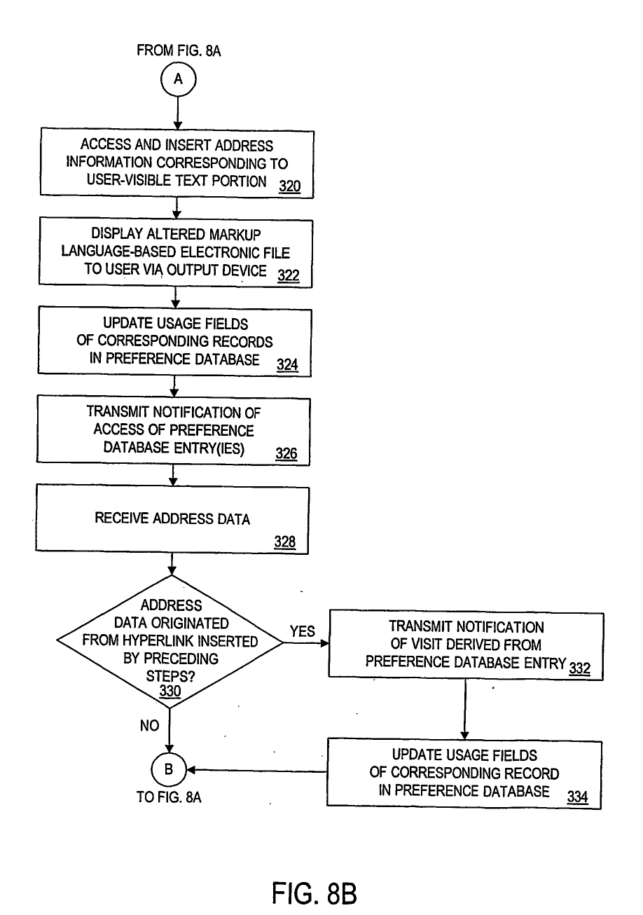

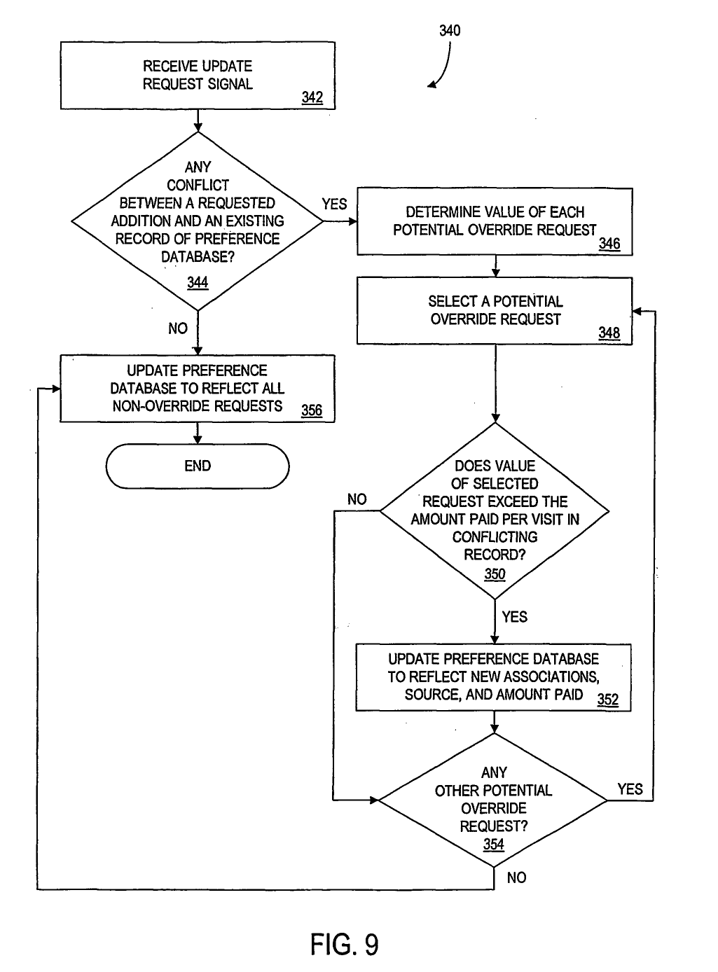

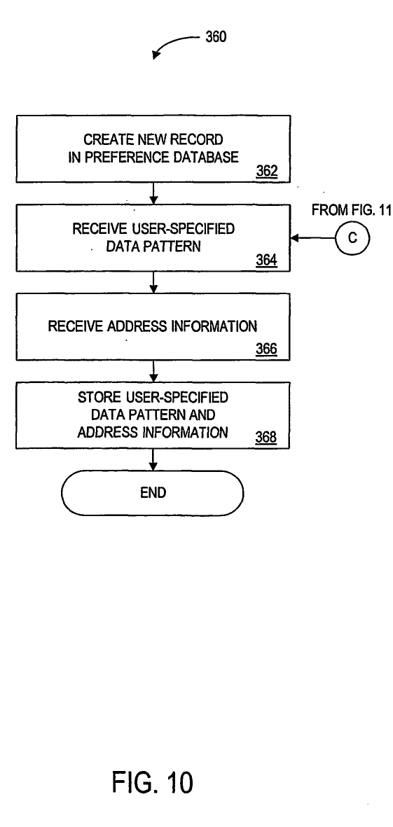

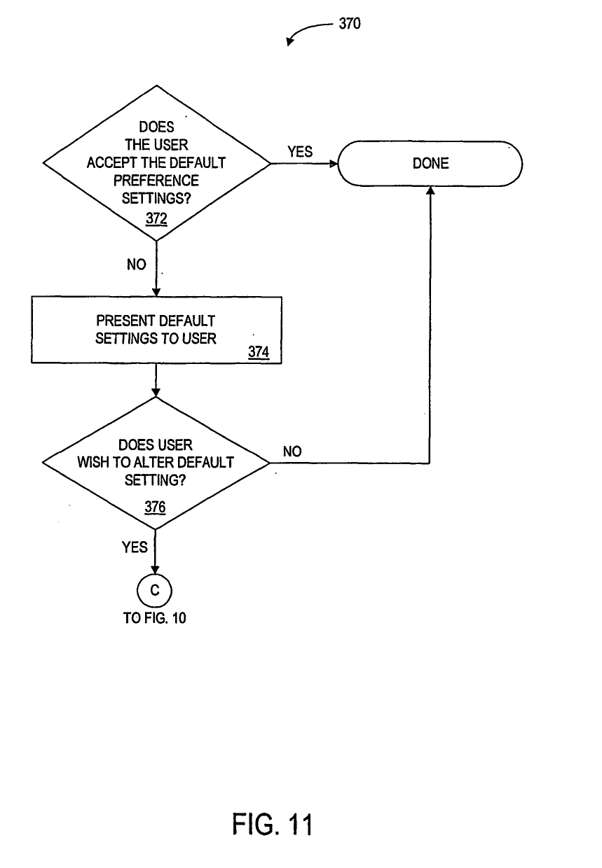

### BACKGROUND

This invention relates to detection of individuals at risk for pathological conditions based on the presence of single nucleotide polymorphisms (SNPs).

During the course of evolution, spontaneous mutations appear in the genomes of organisms. It has been estimated that variations in genomic DNA sequences are created continuously at a rate of about 100 new single base changes per individual (Kondrashow, J. Theor. Biol., 175:583-594, 1995; Crow, Exp. Clin. Immunogenet., 12:121-128, 1995). These changes, in the progenitor nucleotide sequences, may confer an evolutionary advantage, in which case the frequency of the mutation will likely increase, an evolutionary disadvantage in which case the frequency of the mutation is likely to decrease, or the mutation will be neutral. In certain cases, the mutation may be lethal in which case the mutation is not passed on to the next generation and so is quickly eliminated from the population. In many cases, an equilibrium is established between the progenitor and mutant sequences so that both are present in the population. The presence of both forms of the sequence results in genetic variation or polymorphism. Over time, a significant number of mutations can accumulate within a population such that considerable polymorphism can exist between individuals within the population.

Numerous types of polymorphisms are known to exist. Polymorphisms can be created when DNA sequences are either inserted or deleted from the genome, for example, by viral insertion. Another source of sequence variation can be caused by the presence of repeated sequences in the genome variously termed short tandem repeats (STR), variable number tandem repeats (VNTR), short sequence repeats (SSR) or microsatellites. These repeats can be dinucleotide, trinucleotide, tetranucleotide or pentanucleotide repeats. Polymorphism results from variation in the number of repeated sequences found at a particular locus.

By far the most common source of variation in the genome are single nucleotide polymorphisms or SNPs. SNPs account for approximately 90% of human DNA polymorphism (Collins et al., Genome Res., 8:1229-1231, 1998). SNPs are single base pair positions in genomic DNA at which different sequence alternatives (alleles) exist in a population. In addition, the least frequent allele must occur at a frequency of 1% or greater. Several definitions of SNPs exist in the literature (Brooks, Gene, 234:177-186, 1999). As used herein, the term “single nucleotide polymorphism” or “SNP” includes all single base variants and so includes nucleotide insertions and deletions in addition to single nucleotide substitutions (e.g. A→G). Nucleotide substitutions are of two types. A transition is the replacement of one purine by another purine or one pyrimidine by another pyrimidine. A transversion is the replacement of a purine for a pyrimidine or vice versa.

The typical frequency at which SNPs are observed is about 1 per 1000 base pairs (Li and Sadler, Genetics, 129:513-523, 1991; Wang et al., Science, 280:1077-1082, 1998; Harding et al., Am. J. Human Genet., 60:772-789, 1997; Taillon-Miller et al., Genome Res., 8:748-754, 1998). The frequency of SNPs varies with the type and location of the change. In base substitutions, two-thirds of the substitutions involve the C⇄T (G⇄A) type. This variation in frequency is thought to be related to 5-methylcytosine deamination reactions that occur frequently, particularly at CpG dinucleotides. In regard to location, SNPs occur at a much higher frequency in non-coding regions than they do in coding regions.

SNPs can be associated with disease conditions in humans or animals. The association can be direct, as in the case of genetic diseases where the alteration in the genetic code caused by the SNP directly results in the disease condition. Examples of diseases in which single nucleotide polymorphisms result in disease conditions are sickle cell anemia and cystic fibrosis. The association can also be indirect, where the SNP does not directly cause the disease but alters the physiological environment such that there is an increased likelihood that the patient will develop the disease. SNPs can also be associated with disease conditions, but play no direct or indirect role in causing the disease. In this case, the SNP is located close to the defective gene, usually within 5 centimorgans, such that there is a strong association between the presence of the SNP and the disease state. Because of the high frequency of SNPs within the genome, there is a greater probability that a SNP will be linked to a genetic locus of interest than other types of genetic markers.

Disease associated SNPs can occur in coding and non-coding regions of the genome. When located in a coding region, the presence of the SNP can result in the production of a protein that is non-functional or has decreased function. More frequently, SNPs occur in non-coding regions. If the SNP occurs in a regulatory region, it may affect expression of the protein. For example, the presence of a SNP in a promoter region, may cause decreased expression of a protein. If the protein is involved in protecting the body against development of a pathological condition, this decreased expression can make the individual more susceptible to the condition.

Numerous methods exist for the detection of SNPs within a nucleotide sequence. A review of many of these methods can be found in Landegren et al., Genome Res., 8:769-776, 1998. SNPs can be detected by restriction fragment length polymorphism (RFLP)(U.S. Pat. Nos. 5,324,631; 5,645,995). RFLP analysis of the SNPs, however, is limited to cases where the SNP either creates or destroys a restriction enzyme cleavage site. SNPs can also be detected by direct sequencing of the nucleotide sequence of interest. Numerous assays based on hybridization have also been developed to detect SNPs. In addition, mismatch distinction by polymerases and ligases has also been used to detect SNPs.

There is growing recognition that SNPs can provide a powerful tool for the detection of individuals whose genetic make-up alters their susceptibility to certain diseases. There are four primary reasons why SNPs are especially suited for the identification of genotypes which predispose an individual to develop a disease condition. First, SNPs are by far the most prevalent type of polymorphism present in the genome and so are likely to be present in or near any locus of interest. Second, SNPs located in genes can be expected to directly affect protein structure or expression levels and so may serve not only as markers but as candidates for gene therapy treatments to cure or prevent a disease. Third, SNPs show greater genetic stability than repeated sequences and so are less likely to undergo changes which would complicate diagnosis. Fourth, the increasing efficiency of methods of detection of SNPs make them especially suitable for high throughput typing systems necessary to screen large populations.

### SUMMARY

The present inventor has discovered novel single nucleotide polymorphisms (SNPs) associated with the development of various diseases, including end stage renal disease, lung cancer, breast cancer, and prostate cancer. As such, these polymorphisms provide a method for diagnosing a genetic predisposition for the development of these diseases in individuals. Information obtained from the detection of SNPs associated with the development of these diseases is of great value in their treatment and prevention.

Accordingly, one aspect of the present invention provides a method for diagnosing a genetic predisposition for end stage renal disease, lung cancer, breast cancer, or prostate cancer in a subject, comprising obtaining a sample containing at least one polynucleotide from the subject, and analyzing the polynucleotide to detect a genetic polymorphism wherein said genetic polymorphism is associated with an altered susceptibility for end stage renal disease, lung cancer, breast cancer, or prostate cancer. In one embodiment, the polymorphism is located in the TGF-β-RII gene.

Another aspect of the present invention provides an isolated nucleic acid sequence comprising at least 10 contiguous nucleotides from SEQ ID NO: 1, or their complements, wherein the sequence contains at least one polymorphic site associated with a disease and in particular end stage renal disease, lung cancer, breast cancer, or prostate cancer.

Yet another aspect of the invention is a kit for the detection of a polymorphism comprising, at a minimum, at least one polynucleotide of at least 10 contiguous nucleotides of SEQ ID NO: 1, or their complements, wherein the polynucleotide contains at least one polymorphic site associated with end stage renal disease, lung cancer, breast cancer, or prostate cancer.

Yet another aspect of the invention provides a method for treating end stage renal disease, lung cancer, breast cancer, or prostate cancer comprising, obtaining a sample of biological material containing at least one polynucleotide from the subject; analyzing the polynucleotide to detect the presence of at least one polymorphism associated with end stage renal disease, lung cancer, breast cancer, or prostate cancer; and treating the subject in such a way as to counteract the effect of any such polymorphism detected.

Still another aspect of the invention provides a method for the prophylactic treatment of a subject with a genetic predisposition to end stage renal disease, lung cancer, breast cancer, or prostate cancer comprising, obtaining a sample of biological material containing at least one polynucleotide from the subject; analyzing the polynucleotide to detect the presence of at least one polymorphism associated with end stage renal disease, lung cancer, breast cancer, or prostate cancer; and treating the subject.

Further scope of the applicability of the present invention will become apparent from the detailed description and drawings provided below. It should be understood, however, that the following detailed description and examples, while indicating preferred embodiments of the invention, are given by way of illustration only, since various changes and modifications within the spirit and scope of the invention will become apparent to those skilled in the art from the following detailed description.

### DEFINITIONS

nt=nucleotide
bp=base pair
kb=kilobase; 1000 base pairs
ESRD=end-stage renal disease
HTN=hypertension
NIDDM=noninsulin-dependent diabetes mellitus
CRF=chronic renal failure
T-GF=tubulo-glomerular feedback
CRG=compensatory renal growth
MODY=maturity-onset diabetes of the young
RFLP=restriction fragment length polymorphism
MASDA=multiplexed allele-specific diagnostic assay
MADGE=microtiter array diagonal gel electrophoresis
OLA=oligonucleotide ligation assay
DOL=dye-labeled oligonucleotide ligation assay
SNP=single nucleotide polymorphism
PCR=polymerase chain reaction

“polynucleotide” and “oligonucleotide” are used interchangeably and mean a linear polymer of at least 2 nucleotides joined together by phosphodiester bonds and may consist of either ribonucleotides or deoxyribonucleotides.

“sequence” means the linear order in which monomers occur in a polymer, for example, the order of amino acids in a polypeptide or the order of nucleotides in a polynucleotide.

“polymorphism” refers to a set of genetic variants at a particular genetic locus among individuals in a population.

“promoter” means a regulatory sequence of DNA that is involved in the binding of RNA polymerase to initiate transcription of a gene. A “gene” is a segment of DNA involved in producing a peptide, polypeptide, or protein, including the coding region, non-coding regions preceding (“leader”) and following (“trailer”) coding region, as well as intervening non-coding sequences (“introns”) between individual coding segments (“exons”). A promoter is herein considered as a part of the corresponding gene. Coding refers to the representation of amino acids, start and stop signals in a three base “triplet” code. Promoters are often upstream (“5′ to”) the transcription initiation site of the gene.

“gene therapy” means the introduction of a functional gene or genes from some source by any suitable method into a living cell to correct for a genetic defect.

“wild type allele” means the most frequently encountered allele of a given nucleotide sequence of an organism.

“genetic variant” or “variant” means a specific genetic variant which is present at a particular genetic locus in at least one individual in a population and that differs from the wild type.

As used herein the terms “patient” and “subject” are not limited to human beings, but are intended to include all vertebrate animals in addition to human beings.

As used herein the terms “genetic predisposition”, “genetic susceptibility” and “susceptibility” all refer to the likelihood that an individual subject will develop a particular disease, condition or disorder. For example, a subject with an increased susceptibility or predisposition will be more likely than average to develop a disease, while a subject with a decreased predisposition will be less likely than average to develop the disease. A genetic variant is associated with an altered susceptibility or predisposition if the allele frequency of the genetic variant in a population or subpopulation with a disease, condition or disorder varies from its allele frequency in the population without the disease, condition or disorder (control population) or a control sequence (wild type) by at least 1%, preferably by at least 2%, more preferably by at least 4% and more preferably still by at least 8%.

As used herein “isolated nucleic acid” means a species of the invention that is the predominate species present (i.e., on a molar basis it is more abundant than any other individual species in the composition). Preferably, an isolated nucleic acid comprises at least about 50, 80 or 90 percent (on a molar basis) of all macromolecular species present. Most preferably, the object species is purified to essential homogeneity (contaminant species cannot be detected in the composition by conventional detection methods).

As used herein, “allele frequency” means the frequency that a given allele appears in a population.

Abbreviations used herein for nucleotides are the same as those in Table 1 of MPEP section 2422 where a=adenine, g=guanine, c=cytosine, t=thyrnine, u=uracil, r=g or a, y=t/u or c, m=a or c, k=g or t/u, s=g or c, w=a or t/u, b=g or c or t/u, d=a or g or t/u, h=a or c or t/u, v=a or g or c, and n=a or g or c or t/u, unknown, or other.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

TGF-β1 Signalling

Numerous animal and human studies have already linked the progression of renal disease, especially its hallmark pathology of interstitial fibrosis and glomerular sclerosis, to increased signalling by TGF-β1. Signalling by TGF-β1 involves specific binding of the ligand to the type II TGF-β1 receptor (abbreviated as TGFβ-RII), present on the plasma membrane of target cells such as fibroblasts in the case of glomerular and interstitial fibrosis. This receptor-ligand complex then heterodimerizes with the type I TGF-β1 receptor (abbreviated as TGFβ-RI). TGFβ-RI is constitutively active. Like the concentrations of ligand (TGF-β1) and TGFβ-RI, the concentration of TGFβ-RII in the plasma membrane ais likely to be rate-limiting for signalling by TGF-β1. All elements of the pathway appear to be subject to complex regulation.

If the level of TGFβ-RII gene product (i.e., protein) is proportional to the level of mRNA, and the mRNA level is proportional to the transcriptional rate of the gene, then a SNP which disrupts a transcriptional activator site would be expected to decrease both the rate of transcription of the gene and the eventual concentration of TGFβ-RII in the plasma membrane of cells which express this protein. The net effect of such a SNP is expected to be protection against renal failure.

TGF-β1 also inhibits cellular proliferation in a number of cell types. Signalling by TGF-β1 is thus expected to be depressed in individuals with a predisposition to malignancies.

Novel Polymorphisms

The present application provides four single nucleotide polymorphisms (SNPs) in genes associated with end stage renal disease due to NIDDM, lung cancer, breast cancer, or prostate cancer. All four polymorphisms are substitutions found on the TGF-β-RII promoter. The location of these SNPs as well as the wild type and variant nucleotides is summarized in Table 7.

Preparation of Samples

The presence of genetic variants in the above genes or their control regions, or in any other genes that may affect susceptibility to disease is determined by screening nucleic acid sequences from a population of individuals for such variants. The population is preferably comprised of some individuals with the disease, so that any genetic variants that are found can be correlated with disease. The population is also preferably comprised of some individuals that have known risk for the disease. The population should preferably be large enough to have a reasonable chance of finding individuals with the sought-after genetic variant. As the size of the population increases, the ability to find significant correlations between a particular genetic variant and susceptibility to disease also increases. Preferably, the population should have 10 or more individuals.

The nucleic acid sequence can be DNA or RNA. For the assay of genomic DNA, virtually any biological sample containing genomic DNA (e.g. not pure red blood cells) can be used. For example, and without limitation, genomic DNA can be conveniently obtained from whole blood, semen, saliva, tears, urine, fecal material, sweat, buccal cells, skin or hair. For assays using cDNA or mRNA, the target nucleic acid must be obtained from cells or tissues that express the target sequence. One preferred source and quantity of DNA is 10 to 30 ml of anticoagulated whole blood, since enough DNA can be extracted from leukocytes in such a sample to perform many repetitions of the analysis contemplated herein.

Many of the methods described herein require the amplification of DNA from target samples. This can be accomplished by any method known in the art but preferably is by the polymerase chain reaction (PCR). Optimization of conditions for conducting PCR must be determined for each reaction and can be accomplished without undue experimentation by one of ordinary skill in the art. In general, methods for conducting PCR can be found in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990.

Other amplification methods include the ligase chain reaction (LCR) (see, Wu and Wallace, Genomics, 4:560-569, 1989; Landegren et al., Science, 241:1077-1080, 1988), transcription amplification (Kwoh et al., Proc. Natl. Acad Sci. USA, 86:1173-1177, 1989), self-sustained sequence replication (Guatelli et al., Proc. Natl. Acad. Sci. USA, 87:1874-1878, 1990), and nucleic acid based sequence amplification (NASBA). The latter two amplification methods involve isothermal reactions based on isothermal transcription, which produces both single stranded RNA (ssRNA) and double stranded DNA (dsDNA) as the amplification products in a ratio of about 30 or 100 to 1, respectively.

Detection of Polymorphisms

Detection of Unknown Polymorphisms

Two types of detection are contemplated within the present invention. The first type involves detection of unknown SNPs by comparing nucleotide target sequences from individuals in order to detect sites of polymorphism. If the most common sequence of the target nucleotide sequence is not known, it can be determined by analyzing individual humans, animals or plants with the greatest diversity possible. Additionally the frequency of sequences found in subpopulations characterized by such factors as geography or gender can be determined.

The presence of genetic variants and in particular SNPs is determined by screening the DNA and/or RNA of a population of individuals for such variants. If it is desired to detect variants associated with a particular disease or pathology, the population is preferably comprised of some individuals with the disease or pathology, so that any genetic variants that are found can be correlated with the disease of interest. It is also preferable that the population be composed of individuals with known risk factors for the disease. The populations should preferably be large enough to have a reasonable chance to find correlations between a particular genetic variant and susceptibility to the disease of interest. In addition, the allele frequency of the genetic variant in a population or subpopulation with the disease or pathology should vary from its allele frequency in the population without the disease or pathology (control population) or the control sequence (wild type) by at least 1%, preferably by at least 2%, more preferably by at least 4% and more preferably still by at least 8%.

Determination of unknown genetic variants, and in particular SNPs, within a particular nucleotide sequence among a population may be determined by any method known in the art, for example and without limitation, direct sequencing, restriction length fragment polymorphism (RFLP), single-strand conformational analysis (SSCA), denaturing gradient gel electrophoresis (DGGE), heteroduplex analysis (HET), chemical cleavage analysis (CCM) and ribonuclease cleavage.

Methods for direct sequencing of nucleotide sequences are well known to those skilled in the art and can be found for example in Ausubel et al., eds., Short Protocols in Molecular Biology, 3^{rd} ed., Wiley, 1995 and Sambrook et al., Molecular Cloning, 2^{nd} ed., Chap. 13, Cold Spring Harbor Laboratory Press, 1989. Sequencing can be carried out by any suitable method, for example, dideoxy sequencing (Sanger et al., Proc. Natl. Acad. Sci. USA, 74:5463-5467, 1977), chemical sequencing (Maxam and Gilbert, Proc. Natl. Acad. Sci. USA, 74:560-564, 1977) or variations thereof. Direct sequencing has the advantage of determining variation in any base pair of a particular sequence.

RFLP analysis (see, e.g. U.S. Pat. Nos. 5,324,631 and 5,645,995) is useful for detecting the presence of genetic variants at a locus in a population when the variants differ in the size of a probed restriction fragment within the locus, such that the difference between the variants can be visualized by electrophoresis. Such differences will occur when a variant creates or eliminates a restriction site within the probed fragment. RFLP analysis is also useful for detecting a large insertion or deletion within the probed fragment. Thus, RFLP analysis is useful for detecting, e.g., an Alu sequence insertion or deletion in a probed DNA segment.

Single-strand conformational polymorphisms (SSCPs) can be detected in <220 bp PCR amplicons with high sensitivity (Orita et al, Proc. Natl. Acad. Sci. USA, 86:2766-2770, 1989; Warren et al., In: Current Protocols in Human Genetics, Dracopoli et al., eds, Wiley, 1994, 7.4.1-7.4.6.). Double strands are first heat-denatured. The single strands are then subjected to polyacrylamide gel electrophoresis under non-denaturing conditions at constant temperature (i.e. low voltage and long run times) at two different temperatures, typically 4-10° C. and 23° C. (room temperature). At low temperatures (4-10° C.), the secondary structure of short single strands (degree of intrachain hairpin formation) is sensitive to even single nucleotide changes, and can be detected as a large change in electrophoretic mobility. The method is empirical, but highly reproducible, suggesting the existence of a very limited number of folding pathways for short DNA strands at the critical temperature. Polymorphisms appear as new banding patterns when the gel is stained.

Denaturing gradient gel electrophoresis (DGGE) can detect single base mutations based on differences in migration between homo- and heteroduplexes (Myers et al., Nature, 313:495-498, 1985). The DNA sample to be tested is hybridized to a labeled wild type probe. The duplexes formed are then subjected to electrophoresis through a polyacrylamide gel that contains a gradient of DNA denaturant parallel to the direction of electrophoresis. Heteroduplexes formed due to single base variations are detected on the basis of differences in migration between the heteroduplexes and the homoduplexes formed.

In heteroduplex analysis (HET) (Keen et al., Trends Genet.7:5, 1991), genomic DNA is amplified by the polymerase chain reaction followed by an additional denaturing step which increases the chance of heteroduplex formation in heterozygous individuals. The PCR products are then separated on Hydrolink gels where the presence of the heteroduplex is observed as an additional band.

Chemical cleavage analysis (CCM) is based on the chemical reactivity of thymine (T) when mismatched with cytosine, guanine or thymine and the chemical reactivity of cytosine (C) when mismatched with thymine, adenine or cytosine (Cotton et al., Proc. Natl. Acad. Sci. USA, 85:4397-4401, 1988). Duplex DNA formed by hybridization of a wild type probe with the DNA to be examined, is treated with osmium tetroxide for T and C mismatches and hydroxylamine for C mismatches. T and C mismatched bases that have reacted with the hydroxylamine or osmium tetroxide are then cleaved with piperidine. The cleavage products are then analyzed by gel electrophoresis.

Ribonuclease cleavage involves enzymatic cleavage of RNA at a single base mismatch in an RNA:DNA hybrid (Myers et al., Science 230:1242-1246, 1985). A ³²P labeled RNA probe complementary to the wild type DNA is annealed to the test DNA and then treated with ribonuclease A. If a mismatch occurs, ribonuclease A will cleave the RNA probe and the location of the mismatch can then be determined by size analysis of the cleavage products following gel electrophoresis.

Detection of Known Polymorphisms

The second type of polymorphism detection involves determining which form of a known polymorphism is present in individuals for diagnostic or epidemiological purposes. In addition to the already discussed methods for detection of polymorphisms, several methods have been developed to detect known SNPs. Many of these assays have been reviewed by Landegren et al., Genome Res., 8:769-776, 1998 and will only be briefly reviewed here.

One type of assay has been termed an array hybridization assay, an example of which is the multiplexed allele-specific diagnostic assay (MASDA) (U.S. Pat. No. 5,834,181; Shuber et al., Hum. Molec. Genet., 6:337-347, 1997). In MASDA, samples from multiplex PCR are immobilized on a solid support. A single hybridization is conducted with a pool of labeled allele specific oligonucleotides (ASO). Any ASOs that hybridize to the samples are removed from the pool of ASOs. The support is then washed to remove unhybridized ASOs remaining in the pool. Labeled ASOs remaining on the support are detected and eluted from the support. The eluted ASOs are then sequenced to determine the mutation present.

Two assays depend on hybridization-based allele-discrimination during PCR. The TaqMan assay (U.S. Pat. No. 5,962,233; Livak et al., Nature Genet., 9:341-342, 1995) uses allele specific (ASO) probes with a donor dye on one end and an acceptor dye on the other end, such that the dye pair interact via fluorescence resonance energy transfer (FRET). A target sequence is amplified by PCR modified to include the addition of the labeled ASO probe. The PCR conditions are adjusted so that a single nucleotide difference will effect binding of the probe. Due to the 5′ nuclease activity of the Taq polymerase enzyme, a perfectly complementary probe is cleaved during the PCR while a probe with a single mismatched base is not cleaved. Cleavage of the probe dissociates the donor dye from the quenching acceptor dye, greatly increasing the donor fluorescence.

An alternative to the TaqMan assay is the molecular beacons assay (U.S. Pat. No. 5,925,517; Tyagi et al., Nature Biotech., 16:49-53, 1998). In the molecular beacons assay, the ASO probes contain complementary sequences flanking the target specific species so that a hairpin structure is formed. The loop of the hairpin is complimentary to the target sequence while each arm of the hairpin contains either donor or acceptor dyes. When not hybridized to a donor sequence, the hairpin structure brings the donor and acceptor dye close together thereby extinguishing the donor fluorescence. When hybridized to the specific target sequence, however, the donor and acceptor dyes are separated with an increase in fluorescence of up to 900 fold. Molecular beacons can be used in conjunction with amplification of the target sequence by PCR and provide a method for real time detection of the presence of target sequences or can be used after amplification.

High throughput screening for SNPs that affect restriction sites can be achieved by Microtiter Array Diagonal Gel Electrophoresis (MADGE) (Day and Humphries, Anal. Biochem., 222:389-395, 1994). In this assay restriction fragment digested PCR products are loaded onto stackable horizontal gels with the wells arrayed in a microtiter format. During electrophoresis, the electric field is applied at an angle relative to the columns and rows of the wells allowing products from a large number of reactions to be resolved.

Additional assays for SNPs depend on mismatch distinction by polymerases and ligases. The polymerization step in PCR places high stringency requirements on correct base pairing of the 3′ end of the hybridizing primers. This has allowed the use of PCR for the rapid detection of single base changes in DNA by using specifically designed oligonucleotides in a method variously called PCR amplification of specific alleles (PASA) (Sommer et al., Mayo Clin. Proc., 64:1361-1372 1989; Sarker et al., Anal. Biochem. 1990), allele-specific amplification (ASA), allele-specific PCR, and amplification refractory mutation system (ARMS) (Newton et al., Nuc. Acids Res., 1989; Nichols et al., Genomics, 1989; Wu et al., Proc. Natl. Acad. Sci. USA, 1989). In these methods, an oligonucleotide primer is designed that perfectly matches one allele but mismatches the other allele at or near the 3′ end. This results in the preferential amplification of one allele over the other. By using three primers that produce two differently sized products, it can be determined whether an individual is homozygous or heterozygous for the mutation (Dutton and Sommer, BioTechniques,11:700-702, 1991). In another method, termed bi-PASA, four primers are used; two outer primers that bind at different distances from the site of the SNP and two allele specific inner primers (Liu et al., Genome Res., 7:389-398, 1997). Each of the inner primers has a non-complementary 5′ end and form a mismatch near the 3′ end if the proper allele is not present. Using this system, zygosity is determined based on the size and number of PCR products produced.

The joining by DNA ligases of two oligonucleotides hybridized to a target DNA sequence is quite sensitive to mismatches close to the ligation site, especially at the 3′ end. This sensitivity has been utilized in the oligonucleotide ligation assay (Landegren et al., Science, 241:1077-1080, 1988) and the ligase chain reaction (LCR; Barany, Proc. Natl. Acad. Sci. USA, 88:189-193, 1991). In OLA, the sequence surrounding the SNP is first amplified by PCR, whereas in LCR, genomic DNA can be used as a template.

In one method for mass screening for SNPs based on the OLA, amplified DNA templates are analyzed for their ability to serve as templates for ligation reactions between labeled oligonucleotide probes (Samotiaki et al., Genomics, 20:238-242, 1994). In this assay, two allele-specific probes labeled with either of two lanthanide labels (europium or terbium) compete for ligation to a third biotin labeled phosphorylated oligonucleotide and the signals from the allele specific oligonucleotides are compared by time-resolved fluorescence. After ligation, the oligonucleotides are collected on an avidin-coated 96-pin capture manifold. The collected oligonucleotides are then transferred to microtiter wells in which the europium and terbium ions are released. The fluorescence from the europium ions is determined for each well, followed by measurement of the terbium fluorescence.

In alternative gel-based OLA assays, numerous SNPs can be detected simultaneously using multiplex PCR and multiplex ligation (U.S. Pat. No. 5,830,711; Day et al., Genomics, 29:152-162, 1995; Grossman et al., Nuc. Acids Res., 22:4527-4534, 1994). In these assays, allele specific oligonucleotides with different markers, for example, fluorescent dyes, are used. The ligation products are then analyzed together by electrophoresis on an automatic DNA sequencer distinguishing markers by size and alleles by fluorescence. In the assay by Grossman et al., 1994, mobility is further modified by the presence of a non-nucleotide mobility modifier on one of the oligonucleotides.

A further modification of the ligation assay has been termed the dye-labeled oligonucleotide ligation (DOL) assay (U.S. Pat. No. 5,945,283; Chen et al., Genome Res., 8:549-556, 1998). DOL combines PCR and the oligonucleotide ligation reaction in a two-stage thermal cycling sequence with fluorescence resonance energy transfer (FRET) detection. In the assay, labeled ligation oligonucleotides are designed to have annealing temperatures lower than those of the amplification primers. After amplification, the temperature is lowered to a temperature where the ligation oligonucleotides can anneal and be ligated together. This assay requires the use of a thennostable ligase and a thermostable DNA polymerase without 5′ nuclease activity. Because FRET occurs only when the donor and acceptor dyes are in close proximity, ligation is inferred by the change in fluorescence.

In another method for the detection of SNPs termed minisequencing, the target-dependent addition by a polymerase of a specific nucleotide immediately downstream (3′) to a single primer is used to determine which allele is present (U.S. Pat. No. 5,846,710). Using this method, several SNPs can be analyzed in parallel by separating locus specific primers on the basis of size via electrophoresis and determining allele specific incorporation using labeled nucleotides.

Determination of individual SNPs using solid phase minisequencing has been described by Syvanen et al., Am. J Hum. Genet., 52:46-59, 1993. In this method the sequence including the polymorphic site is amplified by PCR using one amplification primer which is biotinylated on its 5′ end. The biotinylated PCR products are captured in streptavidin-coated microtitration wells, the wells washed, and the captured PCR products denatured. A sequencing primer is then added whose 3′ end binds immediately prior to the polymorphic site, and the primer is elongated by a DNA polymerase with one single labeled dNTP complementary to the nucleotide at the polymorphic site. After the elongation reaction, the sequencing primer is released and the presence of the labeled nucleotide detected. Alternatively, dye labeled dideoxynucleoside triphosphates (ddNTPs) can be used in the elongation reaction (U.S. Pat. No. 5,888,819; Shumaker et al., Human Mut., 7:346-354, 1996). In this method, incorporation of the ddNTP is determined using an automatic gel sequencer.

Minisequencing has also been adapted for use with microarrays (Shumaker et al., Human Mut., 7:346-354, 1996). In this case, elongation (extension) primers are attached to a solid support such as a glass slide. Methods for construction of oligonucleotide arrays are well known to those of ordinary skill in the art and can be found, for example, in Nature Genetics, Suppl., Vol. 21, January, 1999. PCR products are spotted on the array and allowed to anneal. The extension (elongation) reaction is carried out using a polymerase, a labeled DNTP and noncompeting ddNTPs. Incorporation of the labeled dNTP is then detected by the appropriate means. In a variation of this method suitable for use with multiplex PCR, extension is accomplished with the use of the appropriate labeled ddNTP and unlabeled ddNTPs (Pastinen et al., Genome Res., 7:606-614, 1997).

Solid phase minisequencing has also been used to detect multiple polymorphic nucleotides from different templates in an undivided sample (Pastinen et al., Clin. Chem., 42:1391-1397, 1996). In this method, biotinylated PCR products are captured on the avidin-coated manifold support and rendered single stranded by alkaline treatment. The manifold is then placed serially in four reaction mixtures containing extension primers of varying lengths, a DNA polymerase and a labeled ddNTP, and the extension reaction allowed to proceed. The manifolds are inserted into the slots of a gel containing formamide which releases the extended primers from the template. The extended primers are then identified by size and fluorescence on a sequencing instrument.

Fluorescence resonance energy transfer (FRET) has been used in combination with minisequencing to detect SNPs (U.S. Pat. No. 5,945,283; Chen et al., Proc. Natl. Acad. Sci. USA, 94:10756-10761, 1997). In this method, the extension primers are labeled with a fluorescent dye, for example fluorescein. The ddNTPs used in primer extension are labeled with an appropriate FRET dye. Incorporation of the ddNTPs is determined by changes in fluorescence intensities.

The above discussion of methods for the detection of SNPs is exemplary only and is not intended to be exhaustive. Those of ordinary skill in the art will be able to envision other methods for detection of SNPs that are within the scope and spirit of the present invention.

In one embodiment the present invention provides a method for diagnosing a genetic predisposition for a disease. In this method, a biological sample is obtained from a subject. The subject can be a human being or any vertebrate animal. The biological sample must contain polynucleotides and preferably genomic DNA. Samples that do not contain genomic DNA, for example, pure samples of mammalian red blood cells, are hot suitable for use in the method. The form of the polynucleotide is not critically important such that the use of DNA, cDNA, RNA or mRNA is contemplated within the scope of the method. The polynucleotide is then analyzed to detect the presence of a genetic variant where such variant is associated with an increased risk of developing a disease, condition or disorder, and in particular end stage renal disease, lung cancer, breast cancer, or prostate cancer. In one embodiment, the genetic variant is located at one of the polymorphic sites contained in Table 7. In another embodiment, the genetic variant is one of the variants contained in Table 7 or the complement of any of the variants contained in Table 7. Any method capable of detecting a genetic variant, including any of the methods previously discussed, can be used. Suitable methods include, but are not limited to, those methods based on sequencing, mini sequencing, hybridization, restriction fragment analysis, oligonucleotide ligation, or allele specific PCR.

The present invention is also directed to an isolated nucleic acid sequence of at least 10 contiguous nucleotides from SEQ ID NO: 1, or the complements of SEQ ID NO 1. In one preferred embodiment, the sequence contains at least one polymorphic site associated with a disease, and in particular end stage renal disease, lung cancer, breast cancer, or prostate cancer. In one embodiment, the polymorphic site is selected from the group contained in Table 7. In another embodiment, the polymorphic site contains a genetic variant, and in particular, the genetic variants contained in Table 7 or the complements of the variants in Table 7. In yet another embodiment, the polymorphic site, which may or may not also include a genetic variant, is located at the 3′ end of the polynucleotide. In still another embodiment, the polynucleotide further contains a detectable marker. Suitable markers include, but are not limited to, radioactive labels, such as radionuclides, fluorophores or fluorochromes, peptides, enzymes, antigens, antibodies, vitamins or steroids.

The present invention also includes kits for the detection of polymorphisms associated with diseases, conditions or disorders, and in particular end stage renal disease, lung cancer, breast cancer, or prostate cancer. The kits contain, at a minimum, at least one polynucleotide of at least 10 contiguous nucleotides of SEQ ID NO 1, or the complements of SEQ ID NO: 1. In one embodiment, the polynucleotide contains at least one polymorphic site, preferably a polymorphic site selected from the group contained in Table 7. Alternatively the 3′ end of the polynucleotide is immediately 5′ to a polymorphic site, preferably a polymorphic site contained in Table 7. In one embodiment, the polymorphic site contains a genetic variant, preferably a genetic variant selected from the group contained in Table 7. In still another embodiment, the genetic variant is located at the 3′ end of the polynucleotide. In yet another embodiment, the polynucleotide of the kit contains a detectable label. Suitable labels include, but are not limited to, radioactive labels, such as radionuclides, fluorophores or fluorochromes, peptides, enzymes, antigens, antibodies, vitamins or steroids.

In addition, the kit may also contain additional materials for detection of the polymorphisms. For example, and without limitation, the kits may contain buffer solutions, enzymes, nucleotide triphosphates, and other reagents and materials necessary for the detection of genetic polymorphisms. Additionally, the kits may contain instructions for conducting analyses of samples for the presence of polymorphisms and for interpreting the results obtained.

In yet another embodiment the present invention provides a method for designing a treatment regime for a patient having a disease, condition or disorder and in particular end stage renal disease, lung cancer, breast cancer, or prostate cancer, caused either directly or indirectly by the presence of one or more single nucleotide polymorphisms. In this method genetic material from a patient, for example, DNA, cDNA, RNA or mRNA is screened for the presence of one or more SNPs associated with the disease of interest. Depending on the type and location of the SNP, a treatment regime is designed to counteract the effect of the SNP.

Alternatively, information gained from analyzing genetic material for the presence of polymorphisms can be used to design treatment regimes involving gene therapy. For example, detection of a polymorphism that either affects the expression of a gene or results in the production of a mutant protein can be used to design an artificial gene to aid in the production of normal, wild type protein or help restore normal gene expression. Methods for the construction of polynucleotide sequences encoding proteins and their associated regulatory elements are well know to those of ordinary skill in the art. Once designed, the gene can be placed in the individual by any suitable means known in the art (Gene Therapy Technologies, Applications and Regulations, Meager, ed., Wiley, 1999; Gene Therapy: Principles and Applications, Blankenstein, ed., Birkhauser Verlag, 1999; Jain, Textbook of Gene Therapy, Hogrefe and Huber, 1998).

The present invention is also useful in designing prophylactic treatment regimes for patients determined to have an increased susceptibility to a disease, condition or disorder, and in particular end stage renal disease, lung cancer, breast cancer, or prostate cancer due to the presence of one or more single nucleotide polymorphisms. In this embodiment, genetic material, such as DNA, cDNA, RNA or mRNA, is obtained from a patient and screened for the presence of one or more SNPs associated either directly or indirectly to a disease, condition, disorder or other pathological condition. Based on this information, a treatment regime can be designed to decrease the risk of the patient developing the disease. Such treatment can include, but is not limited to, surgery, the administration of pharmaceutical compounds or nutritional supplements, and behavioral changes such as improved diet, increased exercise, reduced alcohol intake, smoking cessation, etc.

### EXAMPLES

Position of the single nucleotide polymorphism (SNP) is given according to the numbering scheme in GenBank Accession Number U37070. Thus, all nucleotides will be positively numbered, rather than bear negative numbers reflecting their position upstream from the transcription initiation site, a scheme often used for promoters. The two numbering systems can be easily interconverted, if necessary. GenBank sequences can be found at http://www.ncbi.nlm.nih.gov/

In the following examples, SNPs are written as “reference sequence” (or “wild type”) nucleotide”→“variant nucleotide.” Changes in nucleotide sequences are indicated in bold print. The standard nucleotide abbreviations are used in which A=adenine, C=cytosine, G=guanine, T=thymine, M=A or C, R=A or G. W=A or T, S=C or G, Y=C or T, K=G or T, V=A or C or G, H=A or C or T; D=A or G or T; B=C or G or T; N=A or C or G or T.

### Example 1

### Detection of Novel Polymorphisms by Direct Sequencing of Leukocyte Genomic DNA

Leukocytes were obtained from human whole blood collected with EDTA as an anticoagulent. Blood was obtained from a group of black men, black women, white men, and white women without any known disease. Blood was also obtained from individuals with end stage renal disease, lung cancer, breast cancer, or prostate cancer as indicated in the tables below.

Genomic DNA was purified from the collected leukocytes using standard protocols well known to those of ordinary skill in the art of molecular biology (Ausubel et al., Short Protocol in Molecular Biology, 3^{rd} ed., John Wiley and Sons, 1995; Sambrook et al., Molecular Cloning, Cold Spring Harbor Laboratory Press, 1989; and Davis et al., Basic Methods in Molecular Biology, Elsevier Science Publishing, 1986). One hundred nanograms of purified genomic DNA was used in each PCR reaction.

Standard PCR reaction conditions were used. Methods for conducting PCR are well known in the art and can be found, for example, in U.S. Pat. Nos. 4,965,188, 4,800,159, 4,683,202, and 4,683,195; Ausbel et al., eds., Short Protocols in Molecular Biology, 3^{rd} ed., Wiley, 1995; and Innis et al., eds., PCR Protocols, Academic Press, 1990. Specific primers used are given in the following examples.

PCR reactions were carried out in a total volume of 50 ul containing 10-15 ng leukocyte genomic DNA, 10 pmol of each primer, 200 nM deoxynucleotide triphosphates (dNTPs), 1.25 U Taq polymerase (Qiagen), 1× Qiagen PCR buffer (50 mM KCl, 10 mM Tris-HCl, pH 8.3, 1.5 mM MgCl₂, and 1×“Q” solution (Qiagen). After an initial 3 minutes denaturation at 94° C., 35 cycles were performed consisting of 1 minute denaturation at 94° C., 1 minute hybridization at 55° C., 2 minute extension at 72° C., followed by a final extension step of 5 minutes at 72° C., and 1 minute cooling at 35° C.

Post-PCR clean-up was performed as follows. PCR reactions were cleaned to remove unwanted primer and other impurities such as salts, enzymes, and unincorporated nucleotides that could inhibit sequencing. One of the following clean-up kits was used: Qiaquick-96 PCR Purification Kit (Qiagen) or Multiscreen-PCR Plates (Millipore, discussed below).

When using the Qiaquick protocol, PCR samples were added to the 96-well Qiaquick silica-gel membrane plate and a chaotropic salt, supplied as “PB Buffer,” was then added to each well. The PB Buffer causes DNA to bind to the membrane. The plate was put onto the Qiagen vacuum manifold and vacuum was applied to the plate in order to pull sample and PB Buffer through the membrane. The filtrate was discarded. Next, the samples were washed twice using “PE Buffer.” Vacuum pressure was applied between each step to remove the buffer. Filtrate was similarly discarded after each wash. After the last PE Buffer wash, maximum vacuum pressure was applied to the membrane plate to generate maximum airflow through the membrane in order to evaporate residual ethanol left from the PE Buffer. The clean PCR product was then eluted from the filter using “EB Buffer.” The filtrate contained the cleaned PCR product and was collected. All buffers were supplied as part of the Qiaquick-96 PCR Purification Kit. The vacuum manifold was also purchased from Qiagen for exclusive use with the Qiaquick-96 Purification Kit.

When using the Millipore Multiscreen-PCR Plates, PCR samples were loaded into the wells of the Multiscreen-PCR Plate and the plate was then placed on a Millipore vacuum manifold. Vacuum pressure was applied for 10 minutes, and the filtrate was discarded. The plate was then removed from the vacuum manifold and 100 μl of Milli-Q water was added to each well to rehydrate the DNA samples. After shaking on a plate shaker for 5 minutes, the plate was replaced on the manifold and vacuum pressure was applied for 5 minutes. The filtrate was again discarded. The plate was removed and 60 μl Milli-Q water was added to each well to again rehydrate the DNA samples. After shaking on a plate shaker for 10 minutes, the 60 μl of cleaned PCR product was transferred from the Multiscreen-PCR plate to another 96-well plate by pipetting. The Millipore vacuum manifold was purchased from Millipore for exclusive use with the Multiscreen-PCR plates.

Cycle sequencing was performed on the clean PCR product using an ABI Prism Big Dye Terminator Cycle Sequencing Ready Reaction kit (Perkin-Elmer). For a total volume of 20 μl, the following reagents were added to each well of a 96-well plate: 2.0 μl Terminator Ready Reaction mix, 3.0 μl 5× Sequencing Buffer (ABI), 5-10 μl template (30-90 ng double stranded DNA), 3.2 μM primer (primer used was the forward primer from the PCR reaction), and Milli-Q water to 20 μl total volume. The reaction plate was placed into a Hybaid thermal cycler block and programmed as follows: ×1 cycle: 1 degree/sec thermal ramp to 94° C., 94° C. for 1 min; ×35 cycles: 1 degree/sec thermal ramp to 94° C., then 94° C. for 10 sec, followed by 1 degree/sec thermal ramp to 50° C., then 50° C. for 10 sec, followed by 1 degree/sec thermal ramp to 60° C., then 60° C. for 4 minutes.

The cycle sequencing reaction product was cleaned up to remove the unincorporated dye-labeled terminators that can obscure data at the beginning of the sequence. A precipitation protocol was used. To each sequencing reaction in the 96-well plate 20 μl of Milli-Q water and 60 μl of 100% isopropanol was added. The plate was left at room temperature for at least 20 minutes to precipitate the extension products. The plate was spun in a plate centrifuge (Jouan) at 3,000×g for 30 minutes.

Without disturbing the pellet, the supernatant was discarded by inverting the plate onto several paper tissues (Kimwipes) folded to the size of the plate. The inverted plate, with Kimwipes in place, was placed into the centrifuge (Jouan) and spun at 700×g for 1 minute. The Kimwipes were discarded and the samples were loaded onto a sequencing gel.

Approximately 1 μl of sequencing product was loaded into each well of a 96-lane 5% Long Ranger (FMC single pack) gel. The running buffer consisted of 1×TBE. The glass plates consisted of ABI 48-cm plates for use with a 96-lane 0.4 mm Mylar shark-tooth comb. A semi-automated ABI Prism 377-96 DNA sequencer was used (ABI 377 with 96-lane, Big Dye upgrades). Sequencing run settings were as follows: run module 48E-1200, 8 hr collection time, 2400 V electrophoresis voltage, 50 mA electrophoresis current, 200 W electrophoresis power, CCD offset of 0, gel temperature of 51° C., 40 mW laser power, and CCD gain of 2.

The SEQUENCHER program (Gene Codes Corp., Ann Arbor, Mich.) was used to ensure that only a high-quality sequence was used for allele assignment. The 5′ end of the sequence was trimmed to a maximum of 25%, until there were fewer than 3 ambiguities. The 3′ end was defined as beginning 100 bases after the trimmed 5′ end. The 3′ end was similarly trimmed to remove any sequence containing 3 or more ambiguities in 25 nucleotides. If any ambiguous bases still remained at the 5′ or 3′ end, they were also removed. These settings are considerably stricter than the baseline default settings of the program. Individual sequences were excluded if they revealed less than 85% identity to the reference sequence (“dirty data algorithm,” SEQUENCHER program).

### Example 2

### G to T Transversion at Position 945 of Human TGFβ-RII Promoter

TABLE 1
ALLELE FREQUENCIES

| | G | T |
|---|---|---|
| CONTROL | | |
| Black men (n = 22 chromosomes) | 17 (77%) | 5 (23%) |
| Black women (n = 28 chromosomes) | 28 (100%) | 0 (0%) |
| White men (n = 30 chromosomes) | 28 (93%) | 2 (7%) |
| White women (n = 6 chromosomes) | 4 (67%) | 2 (33%) |
| DISEASE | | |
| BREAST CANCER | | |
| Black women (n = 8 chromosomes) | 8 (100%) | 0 (0%) |
| White women (n = 4 chromosomes) | 4 (100%) | 0 (0%) |
| LUNG CANCER | | |
| Black men (n = 12 chromosomes) | 12 (100%) | 0 (0%) |
| Black women (n = 14 chromosomes) | 14 (100%) | 0 (0%) |
| White men (n = 6 chromosomes) | 6 (100%) | 0 (0%) |
| PROSTATE CANCER | | |
| Black men (n = 6 chromosomes) | 6 (100%) | 0 (0%) |
| White men (n = 12 chromosomes) | 12 (100%) | 0 (0%) |
| ESRD due to NIDDM | | |
| Black men (n = 6 chromosomes) | 6 (100%) | 0 (0%) |
| Black women (n = 6 chromosomes) | 6 (100%) | 0 (0%) |
| White men (n = 6 chromosomes) | 6 (100%) | 0 (0%) |
| White women (n = 6 chromosomes) | 6 (100%) | 0 (0%) |

TABLE 2
GENOTYPE FREQUENCIES

| | G/G | G/T | T/T |
|---|---|---|---|
| CONTROLS | | | |
| Black men (n = 11) | 6 (55%) | 5 (45%) | 0 (0%) |
| Black women (n = 14) | 14 (100%) | 0 (0%) | 0 (0%) |
| White men (n = 15) | 13 (87%) | 2 (13%) | 0 (0%) |
| White women (n = 3) | 1 (33%) | 2 (67%) | 0 (0%) |
| DISEASE | | | |
| BREAST CANCER | | | |
| Black women (n = 4) | 4 (100%) | 0 (0%) | 0 (0%) |
| White women (n = 2) | 2 (100%) | 0 (0%) | 0 (0%) |
| LUNG CANCER | | | |
| Black men (n = 6) | 6 (100%) | 0 (0%) | 0 (0%) |
| Black women (n = 7) | 7 (100%) | 0 (0%) | 0 (0%) |
| White men (n = 3) | 3 (100%) | 0 (0%) | 0 (0%) |
| PROSTATE CANCER | | | |
| Black men (n = 3) | 3 (100%) | 0 (0%) | 0 (0%) |
| White men (n = 6) | 6 (100%) | 0 (0%) | 0 (0%) |
| ESRD due to NIDDM | | | |
| Black men (n = 3) | 3 (100%) | 0 (0%) | 0 (0%) |
| Black women (n = 3) | 3 (100%) | 0 (0%) | 0 (0%) |
| White men (n = 3) | 3 (100%) | 0 (0%) | 0 (0%) |
| White women (n = 3) | 3 (100%) | 0 (0%) | 0 (0%) |

PCR and sequencing were conducted as in Example 1. The sense primer was 5′-GGACATATCTGAAAGAGAAAGGGGG-3′ (SEQ ID NO: 2) and the antisense primer was 5′- TTGGGAGTCACCTGAATGCTTG-3′ (SEQ ID NO: 3). The PCR product produced spanned bases 892 to 1113 of the TGF-β-RII promoter.

As demonstrated above, the control samples all approximate Hardy-Weinberg equilibrium. A frequency of 0.77 for the G allele (“p”) and 0.23 for the T allele (“q”) among black male control individuals predicts genotype frequencies of 59% G/G, 36% G/T, and 5% T/T at Hardy-Weinberg equilibrium (p²+2pq+q²=1). The observed genotype frequencies were 55% G/G, 45% G/T, and 0% T/T, in close agreement with those predicted for Hardy-Weinberg equilibrium.

A frequency of 1.0 for the G allele (“p”) and 0 for the T allele (“q”) among black female control individuals predicts genotype frequencies of 100% G/G, 0% G/T, and 0% T/T at Hardy-Weinberg equilibrium (p²+2pq+q²=1). The observed genotype frequencies were 100% G/G, 0% G/T, and 0% T/T, in perfect agreement with those predicted for Hardy-Weinberg equilibrium.

A frequency of 0.93 for the G allele (“p”) and 0.07 for the T allele (“q”) among white male control individuals predicts genotype frequencies of 86% G/G, 14% G/T, and 0% T/T at Hardy-Weinberg equilibrium (p²+2pq+q²=1). The observed genotype frequencies were 87% G/G, 13% G/T, and 0% T/T, in very close agreement with those predicted for Hardy-Weinberg equilibrium.

A frequency of 0.67 for the G allele (“p”) and 0.33 for the T allele (“q”) among white female control individuals predicts genotype frequencies of 45% G/G, 44% G/T, and 11% T/T at Hardy-Weinberg equilibrium (p²+2pq+q²=1). The observed genotype frequencies were 33% G/G, 67% G/T, and 0% T/T, in fairly close agreement with those predicted for Hardy-Weinberg equilibrium.

The frequency of the G allele, and especially of the G/G genotype, was higher than control frequencies for white women with breast cancer (G allele frequency 100% vs. 67% control; G/G genotype frequency 100% vs. 33% control), black men with lung cancer (G allele frequency 100% vs. 77% control; G/G genotype frequency 100% vs. 55% control), white men with lung cancer (G allele frequency 100% vs. 93% control; G/G genotype frequency 100% vs. 87% control), black men with prostate cancer (G allele frequency 100% vs. 77% control; G/G genotype frequency 100% vs. 55% control), white men with prostate cancer (G allele frequency 100% vs. 93% control; G/G genotype frequency 100% vs. 87% control), black men with ESRD due to NIDDM (G allele frequency 100% vs. 77% control; G/G genotype frequency 100% vs. 55% control), white men with ESRD due to NIDDM (G allele frequency 100% vs. 93% control; G/G genotype frequency 100% vs. 87% control), and white women with ESRD due to NIDDM (G allele frequency 100% vs. 67% control; G/G genotype frequency 100% vs. 33% control).

These data suggest that the reference allele (G) at this locus predisposes white men and women, and black men to the following diseases: breast, lung, and prostate cancer, and ESRD due to NIDDM. In other words, the SNP (T allele) is protective. Black women appear not to have the T allele, so this locus appears to be neutral for them. However, from the data for the other three population groups (white and black men, and white women), it is likely that the T allele predisposes black women to breast and lung cancer, as well as ESRD due to NIDDM.

The G945→T SNP does not disrupt any known transcriptional regulatory site. To be consistent with current models of increased TGFβ1 signalling as a cause of renal failure, and decreased TGFβ1 signalling as a cause of cancer, as yet unknown transcriptional repressor(s) and activator(s) are predicted to bind to this region of the TGFβ-RII promoter.

### Example 3

### G to M (A or C) Substitution at Position 983 of Human TGFβ-RII Promoter

TABLE 3
ALLELE FREQUENCIES

| | G | A | C |
|---|---|---|---|
| CONTROL | | | |
| Black men (n = 22 chromosomes) | 18 (82%) | 4 (18%) | 0 (0%) |
| Black women (n = 30 chromosomes) | 29 (97%) | 1 (3%) | 0 (0%) |
| White men (n = 30 chromosomes) | 30 (100%) | 0 (0%) | 0 (0%) |
| White women (n = 6 chromosomes) | 3 (50%) | 1 (17%) | 2 (33%) |
| DISEASE | | | |
| BREAST CANCER | | | |
| Black women (n = 8 chromosomes) | 8 (100%) | 0 (0%) | 0 (0%) |
| White women (n = 4 chromosomes) | 4 (100%) | 0 (0%) | 0 (0%) |
| LUNG CANCER | | | |
| Black men (n = 12 chromosomes) | 12 (100%) | 0 (0%) | 0 (0%) |
| Black women (n = 14 chromosomes) | 14 (100%) | 0 (0%) | 0 (0%) |
| White men (n = 6 chromosomes) | 4 (67%) | 2 (33%) | 0 (0%) |
| PROSTATE CANCER | | | |
| Black men (n = 6 chromosomes) | 6 (100%) | 0 (0%) | 0 (0%) |
| White men (n = 12 chromosomes) | 12 (100%) | 0 (0%) | 0 (0%) |
| ESRD due to NIDDM | | | |
| Black men (n = 6 chromosomes) | 4 (67%) | 0 (0%) | 2 (33%) |
| Black women (n = 6 chromosomes) | 6 (100%) | 0 (0%) | 0 (0%) |
| White men (n = 6 chromosomes) | 6 (100%) | 0 (0%) | 0 (0%) |
| White women (n = 6 chromosomes) | 6 (100%) | 0 (0%) | 0 (0%) |

TABLE 4
GENOTYPE FREQUENCIES

| | G/G | G/A | A/A | C/C |
|---|---|---|---|---|
| CONTROLS | | | | |
| Black men (n = 11) | 9 (82%) | 0 (0%) | 2 (18%) | 0 (0%) |
| Black women (n = 15) | 14 (93%) | 1 (7%) | 0 (0%) | 0 (0%) |
| White men (n = 15) | 15 (100%) | 0 (0%) | 0 (0%) | 0 (0%) |
| White women (n = 3) | 1 (33%) | 1 (33%) | 0 (0%) | 1 (33%) |
| DISEASE | | | | |
| BREAST CANCER | | | | |
| Black women (n = 4) | 4 (100%) | 0 (0%) | 0 (0%) | 0 (0%) |
| White women (n = 2) | 2 (100%) | 0 (0%) | 0 (0%) | 0 (0%) |
| LUNG CANCER | | | | |
| Black men (n = 6) | 6 (100%) | 0 (0%) | 0 (0%) | 0 (0%) |
| Black women (n = 7) | 7 (100%) | 0 (0%) | 0 (0%) | 0 (0%) |
| White men (n = 3) | 2 (67%) | 0 (0%) | 1 (33%) | 0 (0%) |
| PROSTATE CANCER | | | | |
| Black men (n = 3) | 3 (100%) | 0 (0%) | 0 (0%) | 0 (0%) |
| White men (n = 6) | 6 (100%) | 0 (0%) | 0 (0%) | 0 (0%) |
| ESRD due to NIDDM | | | | |
| Black men (n = 3) | 2 (67%) | 0 (0%) | 0 (0%) | 1 (33%) |
| Black women (n = 3) | 3 (100%) | 0 (0%) | 0 (0%) | 0 (0%) |
| White men (n = 3) | 3 (100%) | 0 (0%) | 0 (0%) | 0 (0%) |
| White women (n = 3) | 3 (100%) | 0 (0%) | 0 (0%) | 0 (0%) |

PCR and sequencing were conducted as in Example 1. The primers were the same as in Example 2. Most SNPs are biallelic, but the G983→M SNP is unusual in being triallelic.

As shown above, the control samples approximate Hiardy-Weinberg equilibrium. A frequency of 0.82 for the G allele (“p”) and 0.18 for the A allele (“q”) among black male control individuals predicts genotype frequencies of 67% G/G, 30% G/A, and 3% A/A at Hardy-Weinberg equilibrium (p²+2pq+q²=1). The observed genotype frequencies were 82% G/G, 0% G/A, and 18% A/A, in distant agreement with those predicted for Hardy-Weinberg equilibrium.

A frequency of 0.97 for the G allele (“p”) and 0.03 for the A allele (“q”) among black female control individuals predicts genotype frequencies of 94% G/G, 6% G/A, and 0% A/A at Hardy-Weinberg equilibrium (p²+2pq+q²=1). The observed genotype frequencies were 100% G/G, 0% G/A, and 0% A/A, in fairly close agreement with those predicted for Hardy-Weinberg equilibrium.

A frequency of 1.0 for the G allele (“p”) and 0 for the A allele (“q”) among white male control individuals predicts genotype frequencies of 100% G/G, 0% G/A, and 0% A/A at Hardy-Weinberg equilibrium(p²+2pq+q²=1). The observed genotype frequencies were 100% G/G, 0% G/A, and 0% A/A, in perfect agreement with those predicted for Hardy-Weinberg equilibrium.

A frequency of 0.50 for the G allele (“pi”), 0.17 for the A allele (“P2”), and 0.33 for the C allele (“p₃”) among white female control individuals predicts genotype frequencies of 25% G/G, 17% G/A, 3% A/A, 11% C/C, 11% A/C, and 33% G/C at Hardy-Weinberg equilibrium. These frequencies can be obtained by expanding the expression (p₁A₁+p₂A₂+p₃A₃)², where p₁+p₂+p₃=1 (Daniel L. Hartl, A Primer of Population Genetics, 2nd ed., Sinauer Associates, Inc., 35, 1988). In this case, allele A₁=G, A₂=A, and A₃=C. The genotype frequencies of A₁A₁ (here, G/G), A₁A₂ (here, G/A), A₂A₂ (here, A/A), A₁A₃ (here, G/C), A₂A₃ (here, A/C), and A₃A₃ (here, C/C) are predicted to be p₁², 2p₁p₂, p₂², 2p₁p₃, 2p₂p₃, and p₃², respectively. The observed genotype frequencies were 33% G/G, 33% G/A, 0% A/A, and 33% C/C, in rather distant agreement with those predicted for Hardy-Weinberg equilibrium.

Assuming as a general rule that a difference in allele or genotype frequency of at least 10% is clinically significant, the following observations can be made. The reference G allele at this locus is increased in frequency relative to the control group, as is the G/G genotype, for white women with breast cancer, black men with lung cancer, black men with prostate cancer, and white women with ESRD due to NIDDM. These data suggest that the G allele predisposes individuals to the above diseases for the above population groups. The G allele is decreased in frequency relative to controls for white men with lung cancer and black men with ESRD due to NIDDM; in the last group, there is the appearance of an otherwise unusual C allele.

This locus appears to be neutral in effect (i.e., possess unchanged allele and genotype frequencies, relative to control individuals) for black women with breast cancer or lung cancer, white men with prostate cancer, and black women and white men with ESRD due to NEDDM.

The G983→M SNP is predicted to disrupt a potential binding site for RFX1_{—}02 (X-box binding regulatory factor or RFX1; an X-box consists of DNA of the sequence 5′-GTNRCC (0-3N)RGYAAC-3′ (SEQ ID NO. 4), (where N is any nucleotide, R is a purine [A or G], and Y is a pyrimidine[C or T]). The 3′ terminus of this binding site ends at nucleotide 972 on the (−) strand. The consensus RFX1_{—}02 binding site consists of the sequence complementary to 5′-NNGTTRCYNNNGYNACNN-3′ (SEQ ID NO.5). Both the G983→A and G983→C forms of this triallelic SNP replace the indicated G in the core recognition sequence. RFX1_{—}02 binding sites occur somewhat frequently, 0.95 matches per 1000 base pairs of random genomic sequence in vertebrates.

Transcriptional regulation by RFX1 can be either positive or negative. An example of transcriptional repression mediated by RFX1 occurs when RFX1 binds to a methylated site near the transcription initiation site of the collagen alpha2(I) gene (Sengupta P K et al., J. Biol. Chem. 274(51):36649-36655, 1999). Conversely, RFX activates expression of major histocompatibility complex (MHC) class II genes; absence of RFX5 results in bare lymphocyte syndrome (Brickey W J, et al., J. Immunol. 163(12):6622-6630, 1999).

Besides being triallelic, the G983→M SNP is additionally complex. The reference allele, G, is increased in frequency in some diseases but decreased in others.

The frequency of the G allele is increased in breast cancer in white women, lung cancer in black men, and prostate cancer in black men. Without being bound by theory, if one assumes that cancer results from inappropriately low TGF-1 signalling, presumably due in part to decreased transcription of the TGF -RII gene, then it follows that RFX acts normally to repress transcription of the TGF -RII gene in these diseases and subpopulations. Replacement of the G by another allele (A or C) would result in less repression of the TGF -RII gene. Put another way, the presence of the reference G allele would result in increased repression of the TGF -RII gene and hence less signalling by TGF-1.

Where the frequency of the G allele is decreased relative to controls, as in white men with lung cancer, consistency with the theory that decreased signalling by TGF-1 underlies cancer would suggest that RFX acts as a transcriptional activator of the TGF RII gene, rather than as a repressor.

The converse is predicted for ESRD due to NIDDM, a condition assumed to result from increased, rather than decreased, signalling by TGF-1. Black men with this disease, in whom the G allele frequency is decreased, suggest that RFX may act as a transcriptional repressor normally, by the same arguments as above. White women with ESRD due to NIDDM, however, in whom the frequency of the G allele is increased relative to that of control individuals, would predict that RFX normally acts as a transcriptional activator in this subpopulation.

### Example 4

### G to W(A or T) Substitution at Position 1009 of Human TGFβ-RII Promoter

TABLE 5
ALLELE FREQUENCIES

| | G | A | T |
|---|---|---|---|
| CONTROL | | | |
| Black men (n = 20 chromosomes) | 10 (50%) | 10 (50%) | 0 (0%) |
| Black women (n = 30 chromosomes) | 9 (30%) | 21 (70%) | 0 (0%) |
| White men (n = 30 chromosomes) | 24 (80%) | 6 (20%) | 0 (0%) |
| White women (n = 6 chromosomes) | 4 (67%) | 2 (33%) | 0 (0%) |
| DISEASE | | | |
| BREAST CANCER | | | |
| Black women (n = 8 chromosomes) | 3 (38%) | 5 (63%) | 0 (0%) |
| White women (n = 4 chromosomes) | 3 (75%) | 1 (25%) | 0 (0%) |
| LUNG CANCER | | | |
| Black men (n = 12 chromosomes) | 2 (17%) | 10 (83%) | 0 (0%) |
| Black women (n = 14 chromosomes) | 2 (14%) | 12 (86%) | 0 (0%) |
| White men (n = 6 chromosomes) | 6 (100%) | 0 (0%) | 0 (0%) |
| PROSTATE CANCER | | | |
| Black men (n = 6 chromosomes) | 1 (17%) | 5 (83%) | 0 (0%) |
| White men (n = 12 chromosomes) | 10 (83%) | 2 (17%) | 0 (0%) |
| ESRD due to NIDDM | | | |
| Black men (n = 6 chromosomes) | 0 (0%) | 4 (67%) | 2 (33%) |
| Black women (n = 6 chromosomes) | 3 (50%) | 3 (50%) | 0 (0%) |
| White men (n = 6 chromosomes) | 4 (67%) | 2 (33%) | 0 (0%) |
| White women (n = 6 chromosomes) | 4 (67%) | 0 (0%) | 2 (33%) |

TABLE 6
GENOTYPE FREQUENCIES

| | G/G | G/A | A/A | T/T |
|---|---|---|---|---|
| CONTROLS | | | | |
| Black men (n = 10) | 3 (30%) | 4 (40%) | 3 (30%) | 0 (0%) |
| Black women (n = 15) | 2 (13%) | 5 (33%) | 8 (53%) | 0 (0%) |
| White men (n = 15) | 10 (67%) | 4 (27%) | 1 (7%) | 0 (0%) |
| White women (n = 3) | 1 (33%) | 2 (67%) | 0 (0%) | 0 (0%) |
| DISEASE | | | | |
| BREAST CANCER | | | | |
| Black women (n = 4) | 1 (25%) | 1 (25%) | 2 (50%) | 0 (0%) |
| White women (n = 2) | 1 (50%) | 1 (50%) | 0 (0%) | 0 (0%) |
| LUNG CANCER | | | | |
| Black men (n = 6) | 0 (0%) | 2 (33%) | 4 (67%) | 0 (0%) |
| Black women (n = 7) | 0 (0%) | 2 (29%) | 5 (71%) | 0 (0%) |
| White men (n = 3) | 3 (100%) | 0 (0%) | 0 (0%) | 0 (0%) |
| PROSTATE CANCER | | | | |
| Black men (n = 3) | 0 (0%) | 1 (33%) | 2 (67%) | 0 (0%) |
| White men (n = 6) | 4 (67%) | 2 (33%) | 0 (0%) | 0 (0%) |
| ESRD due to NTDDM | | | | |
| Black men (n = 3) | 0 (0%) | 0 (0%) | 2 (67%) | 1 (33%) |
| Black women (n = 3) | 1 (33%) | 1 (33%) | 1 (33%) | 0 (0%) |
| White men (n = 3) | 1 (33%) | 2 (67%) | 0 (0%) | 0 (0%) |

| | | |
|---|---|---|
| White women (n = 3) | 1 (33%) | G/T = 2 (67%) |

PCR and sequencing were conducted as in Example 1. The primers were the same as in Example 2. Most SNPs are biallelic, but the G1009→W SNP is unusual in being triallelic.

As show above, the control samples approximate Hardy-Weinberg equilibrium. A frequency of 0.50 for the G allele (“p”) and 0.50 for the A allele (“q”) among black male control individuals predicts genotype frequencies of 25% G/G, 50% G/A, and 25% A/A at Hardy-Weinberg equilibrium (p²+2pq+q²=1). The observed genotype frequencies were 30% G/G, 40% G/A, and 30% A/A, in close agreement with those predicted for Hardy-Weinberg equilibrium.

A frequency of 0.30 for the G allele (“p”) and 0.70 for the A allele (“q”) among black female control individuals predicts genotype frequencies of 9% G/G, 42% G/A, and 49% A/A at Hardy-Weinberg equilibrium (p²+2pq+q²=1). The observed genotype frequencies were 13% G/G, 33% G/A, and 53% A/A, in reasonably close agreement with those predicted for Hardy-Weinberg equilibrium.

A frequency of 0.80 for the G allele (“p”) and 0.20 for the A allele (“q”) among white male control individuals predicts genotype frequencies of 64% G/G, 32% G/A, and 4% A/A at Hardy-Weinberg equilibrium (p²+2pq+q²=1). The observed genotype frequencies were 67% G/G, 27% G/A, and 7% A/A, in close agreement with those predicted for Hardy-Weinberg equilibrium.

A frequency of 0.67 for the G allele (“p”) and 0.33 for the A allele (“q”) among white female control individuals predicts genotype frequencies of 45% G/G, 44% G/A, and 11% A/A at Hardy-Weinberg equilibrium (p²+2pq+q²=1). The observed genotype frequencies were 33% G/G, 67% G/A, and 0% A/A, in fair agreement with those predicted for Hardy-Weinberg equilibrium.

Assuming as a general rule that a difference in allele or genotype frequency of at least 10% is clinically significant, the following observations can be made. For black women with breast cancer, the frequency of the G allele was increased relative to controls, suggesting that the reference G allele contributes to breast cancer in black women. The frequency of the G/G genotype was increased and the G/A genotype decreased relative to controls, and also relative to that expected for Hardy-Weinberg equilibrium.

The G allele frequency for black women with breast cancer was 38%, vs. 30% in controls. The expected genotype distribution according to Hardy-Weinberg equilibrium was 9% G/G, 42% G/A, and 49% A/A for black women. However, black women with breast cancer had a genotype frequency of 25% G/G, almost three times higher than the 9% frequency expected, and twice the 13% observed in the control group. The frequency of the G/A genotype was only 25% among black women with breast cancer, as compared to 42% predicted for Hardy-Weinberg equilibrium, and 33% observed in controls.

For white women with breast cancer, the G allele frequency was less markedly increased than among black women: 75%, as compared to 67% in controls. Conversely, the frequency of the A allele was slightly decreased, from 33% in controls to 25% among white women with breast cancer. The expected genotype distribution according to Hardy-Weinberg equilibrium was 45% G/G, 44% G/A, and 11% A/A. The distribution of genotypes for white women with breast cancer was 50% G/G, 50% G/A, 0% A/A, again showing a slight excess of G/G and G/A genotypes at the expense of the A/A genotype. These data suggest that the G allele also predisposes white women to breast cancer, although not to the same degree as black women.

For white men with lung cancer, the situation is similar to breast cancer. White men with lung cancer have a marked increase in the frequency of the reference G allele relative to controls, 100% vs. 80%. The distribution of genotypes for white men with lung cancer (100% G/G) in no way resembles the predicted Hardy-Weinberg distribution (64% G/G, 32% G/A, 4% A/A), nor the observed distribution among control individuals (67% G/G, 27% G/A, 7% A/A). These data suggest that the G allele strongly predisposes white men to lung cancer.

The story is different for African-Arnericans with lung cancer. Both black men and women have a markedly decreased frequency of the G allele relative to control, 0% vs. 50% for black male controls and 30% for black female controls. Conversely, the frequency of the A allele is increased among black men and women with lung cancer. This can best be seen by looking at the frequency of the A/A genotype. It is 67% in black men with lung cancer, more than twice as much as the 25% predicted for black men at Hardy-Weinberg equilibrium, and the 30% observed among black male controls. Similarly, the frequency of the A/A genotype is 71% among black women with lung cancer, as compared to only 49% predicted for black women at Hardy-Weinberg equilibrium, and the 53% observed among black female controls. These data suggest that the A allele strongly predisposes black men and women to lung cancer.

For prostate cancer, the deviation from control allele frequencies is much more marked for black men than white men. The G allele frequency is decreased nearly three-fold among black men with prostate cancer, 17%, as compared to 50% for control individuals. The frequency of the G/G genotype is reduced to 0% for black men with prostate cancer, as compared to 25% predicted by Hardy-Weinberg equilibrium, and 30% observed among control individuals. These data suggest that the G allele is protective against prostate cancer in black men, or alternatively, that the A allele predisposes to prostate cancer in black men. The frequency of the A/A genotype is 67% among black patients, over twice the A/A frequency predicted for Hardy-Weinberg equilibrium (25%) as well as that observed among control individuals (30%). For white men with prostate cancer, the allele and genotype frequencies are essentially the same as control.

For black and white men with ESRD due to NIDDM, the frequency of the G allele is markedly decreased relative to control, suggesting that the G allele is protective against this disease in men. The G allele frequency is 0% for black men with ESRD due to NIDDM, vs. 50% for control individuals. The A allele, on the other hand, has a frequency of 67% among black men with ESRD due to NIDDM, vs. 50% among controls. A second SNP, the T allele at position 1009 in the TGF RII promoter, which does not occur at all in the control group, is present at a frequency of 33% among black men with ESRD due to NIDDM. The A and T alleles, therefore, appear to confer predisposition to ESRD due to NIDDM for black men.

White men with ESRD due to NIDDM similarly have over a two-fold lower frequency of the reference G allele compared to control individuals, 33% vs. 80%, suggesting that the G allele is protective against disease for white men. White men with ESRD due to NIDDM did not have the T allele; the A allele appears to be the major disease-predisposing allele for white men.

Black women with ESRD due to NIDDM have a higher frequency of the G allele, 50% relative to control individuals whose G allele frequency is only 30%. The G allele appears to strongly predispose black women to ESRD due to NIDDM, in contrast to the protective effect of the G allele for white and black men.

White women with ESRD due to NIDDM, like black men with the disease, have a 33% frequency of the T allele. The T allele does not appear at all among control individuals. Thus, the T allele strongly predispose white women to ESRD due to NIDDM.

The GI009→W SNP does not disrupt any known transcriptional regulatory site. Control at this site is expected to be extremely complex, involving both activator(s) and repressor(s) of transcription, since the reference allele (G) can either contribute to, or protect against, disease depending on ethnicity (e.g. black vs. white men with lung cancer) or gender (e.g. black men vs. women with ESRD due to NIDDM).

TABLE 7

| Gene | Region | Location | Wild Type | Variant | SEQ ID |
|---|---|---|---|---|---|
| TGFβ-RII | Promoter | 945 | G | T | 1 |
| | | 983 | G | M | 1 |
| | | 1009 | G | W | 1 |

Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may fuinction, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

1. A method for diagnosing a genetic susceptibility for a disease, condition, or disorder in a subject comprising:
obtaining a biological sample containing nucleic acid from said subject; and
analyzing said nucleic acid to detect the presence or absence of a single nucleotide polymorphism in the TGFβ-RII gene, wherein said single nucleotide polymorphism is associated with a genetic predisposition for a disease, condition or disorder selected from the group consisting of end stage renal disease, lung cancer, breast cancer, and prostate cancer.

2. The method of claim 1, wherein the gene TGFβ-RII comprises SEQ ID NO: 1.

3. The method of claim 1, wherein said nucleic acid is DNA, RNA, cDNA or mRNA.

4. The method of claim 2, wherein said single nucleotide polymorphism is located at position 945, 983 or 1009 of SEQ ID NO: 1.

5. The method of claim 4, wherein said single nucleotide polymorphism is selected from the group consisting of G945→T, G983→M, and G1009→W and the complements thereof namely C945→A, C983→K, and C1009→W.

5. The method of claim 1, wherein said analysis is accomplished by sequencing, mini sequencing, hybridization, restriction fragment analysis, oligonucleotide ligation assay or allele specific PCR.

6. An isolated polynucleotide comprising at least 10 contiguous nucleotides of SEQ ID NO: 1, or the complement thereof, and containing at least one single nucleotide polymorphism at position 945, 983, or 1009 of SEQ ID NO: 1 wherein said at least one single nucleotide polymorphism is associated with a 5 disease, condition or disorder selected from the group consisting of end stage renal disease, lung cancer, breast cancer, and prostate cancer.

7. The isolated polynucleotide of claim 7, wherein at least one single nucleotide polymorphism is selected from the group consisting of G945→T, G983→M, and G1 009→W and the complements thereof namely C945→A, C983→K, and C1009→W.

8. The isolated polynucleotide of claim 7, wherein said at least one single nucleotide polymorphism is located at the 3′ end of said nucleic acid sequence.

9. The isolated polynucleotide of claim 7, further comprising a detectable label.

10. The isolated nucleic acid sequence of claim 10, wherein said detectable label is selected from the group consisting of radionuclides, fluorophores or fluorochromes, peptides, enzymes, antigens, antibodies, vitamins or steroids.

11. A kit comprising at least one isolated polynucleotide of at least 10 contiguous nucleotides of SEQ ID NO: 1 or the complement thereof, and containing at least one single nucleotide polymorphism associated with a disease, condition, or disorder selected from the group consisting of end stage renal disease, lung cancer, breast cancer, and prostate cancer; and instructions for using said polynucleotide for detecting the presence or absence of said at least one single nucleotide polymorphism in said nucleic acid.

12. The kit of claim 12 wherein said at least one single nucleotide polymorphism is located at position 945, 983, or 1009 of SEQ ID NO: 1.

13. The kit of claim 13 wherein said at least one single nucleotide polymorphism is selected from the group consisting of G945→T, G983→M, and G1009→W and the complements thereof namely C945→A, C983→K, and C1009→W.

14. The kit of claim 12, wherein said single nucleotide polymorphism is located at the 3′ end of said polynucleotide.

15. The kit of claim 12, wherein said polynucleotide further comprises at least one detectable label.

16. The kit of claim 16, wherein said label is chosen from the group consisting of radionuclides, fluorophores or fluorochromes, peptides enzymes, antigens, antibodies, vitamins or steroids.

17. A kit comprising at least one polynucleotide of at least 10 contiguous nucleotides of SEQ ID NO: 1 or the complement thereof, wherein the 3′ end of said polynucleotide is immediately 5′ to a single nucleotide polymorphism site associated with a genetic predisposition to disease, condition, or disorder selected from the group consisting of end stage renal disease, lung cancer, breast cancer, and prostate cancer; and instructions for using said polynucleotide for detecting the presence or absence of said single nucleotide polymorphism in a biological sample containing nucleic acid.

18. The kit of claim 18, wherein said single nucleotide polymorphism site is located at position 945, 983 or 1009 of SEQ ID NO: 1.

19. The kit of claim 19, wherein said at least one polynucleotide further comprises a detectable label.

20. The kit of claim 20, wherein said detectable label is chosen from the group consisting of radionuclides, fluorophores or fluorochromes, peptides, enzymes, antigens, antibodies, vitamins or steroids.

21. A method for treatment or prophylaxis in a subject comprising:
obtaining a sample of biological material containing nucleic acid from a subject;
analyzing said nucleic acid to detect the presence or absence of at least one single nucleotide polymorphism in SEQ ID NO: 1 or the complement thereof associated with a disease, condition, or disorder selected from the group consisting of end stage renal disease, lung cancer, breast cancer, and prostate cancer; and
treating said subject for said disease, condition or disorder.

22. The method of claim 22 wherein said nucleic acid is selected from the group consisting of DNA, cDNA, RNA and mRNA.

23. The method of claim 22, wherein said at least one single nucleotide polymorphism is located at position 945, 983, or 1009 of SEQ ID NO: 1.

24. The method of claim 22 wherein said at least one single nucleotide polymorphism is selected from the group consisting of G945→T, G983→M, and G1009→W and the complements thereof namely C945→A, C983→K, and C1009→W.

25. The method of claim 22 wherein said treatment counteracts the effect of said at least one single nucleotide polymorphism detected.
